(19) United States
(12) Patent Application Publication
(10) Pub. No.: US 2023/0008824 A1
(43) Pub. Date: Jan. 12, 2023

(54) Dental Diagnostic Assembly

(71) Applicant: Carl Hamil, Alba, TX (US)

(72) Inventor: Carl Hamil, Alba, TX (US)

(21) Appl. No.: 17/370,105

(22) Filed: Jul. 8, 2021

(51) Int. Cl. A61B 5/0534; A61B 5/00

(52) U.S. Cl. CPC A61B 5/0534; A61B 5/742

(57) ABSTRACT

A dental diagnostic assembly for diagnosing dental health includes a handle that can be gripped by a dentist. A sensing probe is coupled to the handle and the sensing probe has a curved end which can be positioned against a tooth in a patient's mouth. A diagnostic unit is in remote communicates a diagnostic pulse to the sensing probe to transmit the diagnostic pulse into the tooth. The diagnostic unit analyzes the resistance to the diagnostic pulse to determine the health of the tooth. Moreover, the diagnostic unit includes a display for displaying numbers indicating the value of the magnitude of resistance to visually communicate the health of the tooth to the dentist.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to diagnostic devices and more particularly pertains to a new diagnostic device for diagnosing dental health. The device includes a sensing probe that can be positioned against a tooth and a diagnostic unit that is in communication with the sensing probe. The diagnostic unit transmits a diagnostic pulse into the sensing probe and the diagnostic unit measures the tooth's reaction to the diagnostic pulse. The diagnostic unit includes a display which displays a number corresponding to the magnitude of the tooth's reaction for diagnosing the health of the tooth.

### (2) Description of Related Art including Information Disclosed Under 37 CFR 1.97 and 1.98.

The prior art relates to diagnostic devices including a percussion instrument for delivering a percussive strike to a tooth and measuring rebound of a striking implement to diagnose the health of the tooth. The prior art discloses a tooth probe which is positioned against a tooth to deliver a constant sinusoidal force to the tooth for diagnosing the health of the tooth. The prior art discloses a vibratory device for imparting an oscillating signal into a tooth to measure the reaction of the tooth for diagnosing health of the tooth. The prior art discloses a tooth diagnosis method that includes a vibration sensor that is positioned against a tooth and a hammer that is employed to strike the tooth for diagnosing health of the tooth.

### BRIEF SUMMARY OF THE INVENTION

An embodiment of the disclosure meets the needs presented above by generally comprising a handle that can be gripped by a dentist. A sensing probe is coupled to the handle and the sensing probe has a curved end which can be positioned against a tooth in a patient's mouth. A diagnostic unit is in remote communicates a diagnostic pulse to the sensing probe to transmit the diagnostic pulse into the tooth. The diagnostic unit analyzes the resistance to the diagnostic pulse to determine the health of the tooth. Moreover, the diagnostic unit includes a display for displaying numbers indicating the value of the magnitude of resistance to visually communicate the health of the tooth to the dentist.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a dental diagnostic assembly according to an embodiment of the disclosure.

FIG. 2 is a front view of an embodiment of the disclosure.

FIG. 3 is a back phantom view of an embodiment of the disclosure.

FIG. 4 is a right side view of an embodiment of the disclosure.

FIG. 5 is a perspective in-use view of an embodiment of the disclosure.

### DETAILED DESCRIPTION OF THE INVENTION

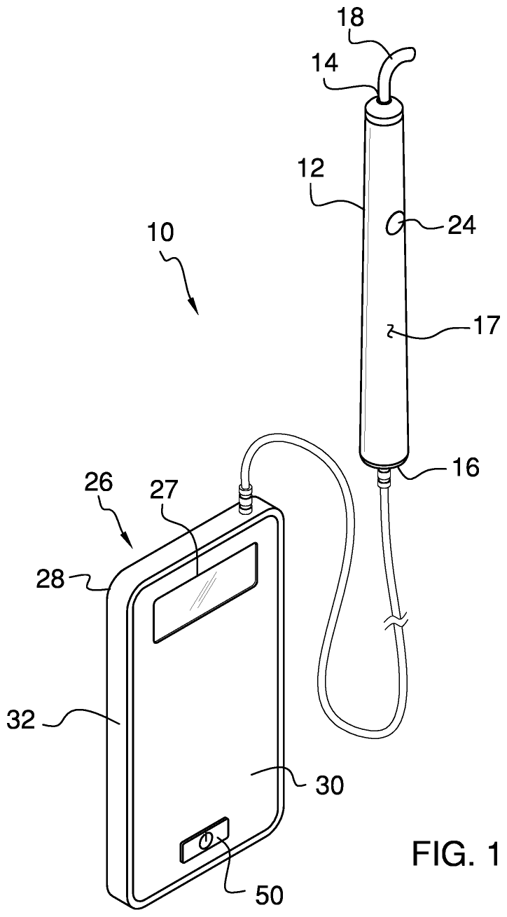

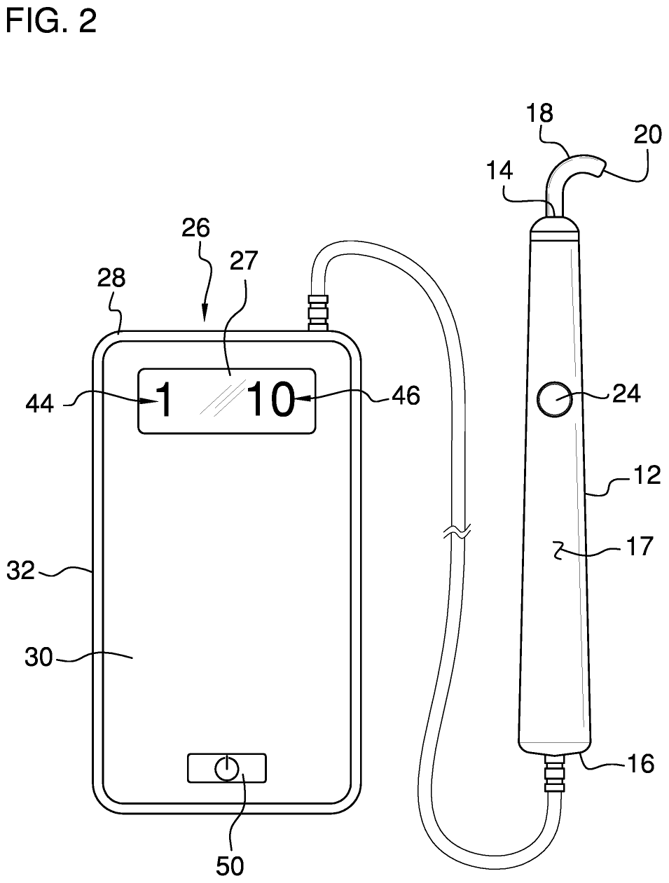

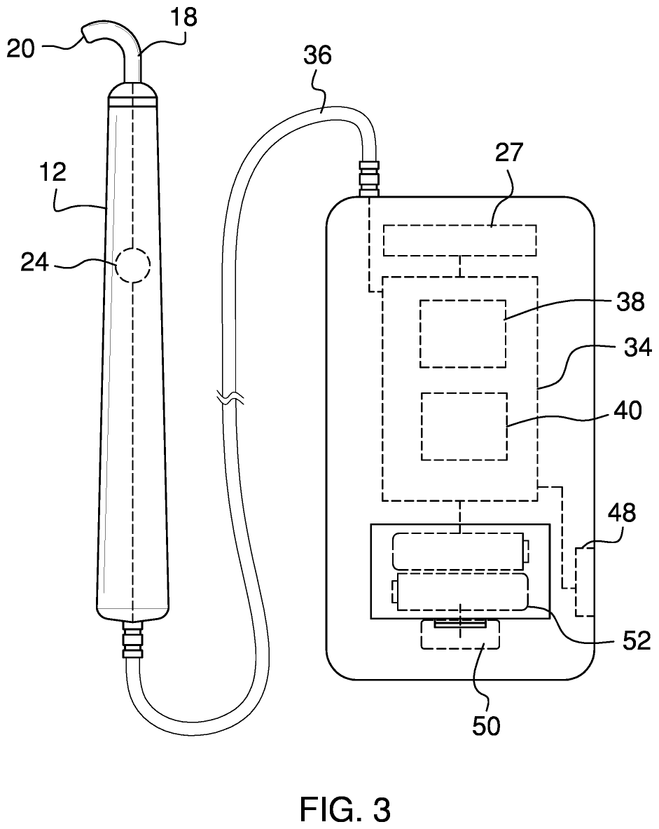

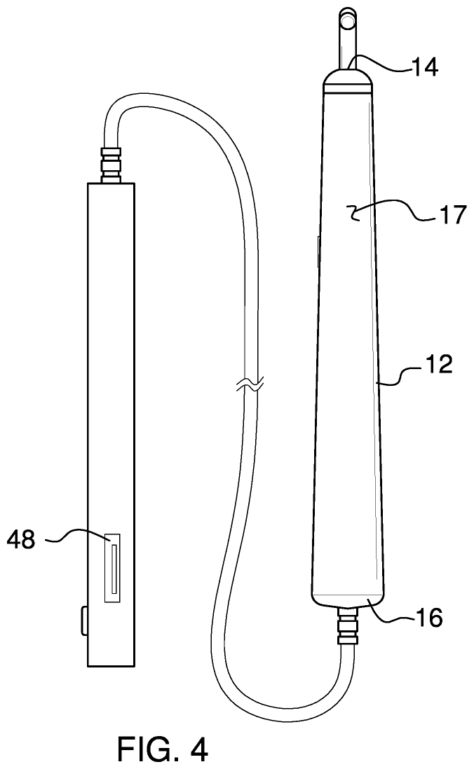

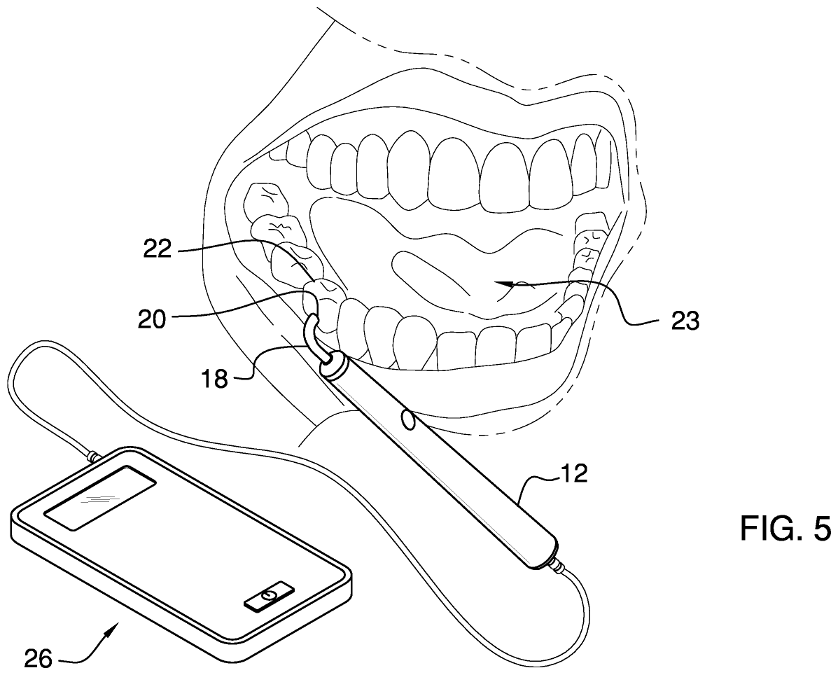

With reference now to the drawings, and in particular to FIGS. 1 through 5 thereof, a new diagnostic device embodying the principles and concepts of an embodiment of the disclosure and generally designated by the reference numeral 10 will be described.

As best illustrated in FIGS. 1 through 5, the dental diagnostic assembly 10 generally comprises a handle 12 that can be gripped by a dentist. The handle 12 has a first end 14, a second end 16 and an outer surface 17 extending between the first end 14 and the second end 16, and the handle 12 is elongated between the first end 14 and the second end 16. The handle 12 may widen between the first end 14 and the second end 16. A sensing probe 18 is coupled to the handle 12 and the sensing probe 18 has a curved end 20. In this way the curved end 20 can be positioned against a tooth 22 in a patient's mouth 23 when the handle 12 is gripped. The sensing probe 18 extends away from the first end 14 of the handle 12 and the curved end 20 is distally positioned with respect to the first end 14 of the handle 12. The sensing probe 18 is comprised of a rigid material, including but not being limited to, steel or ceramic, thereby facilitating the sensing probe 18 to transmit a force. In this way the sensing probe 18 can to transmit the force into the tooth 22 when the sensing probe 18 is positioned against the tooth 22. An activate button 24 is movably integrated into the outer surface 17 of the handle 12 and the activate button 24 can be manipulated by the dentist.

A diagnostic unit 26 is provided and the diagnostic unit 26 is in remote communication with the sensing probe 18. The diagnostic unit 26 produces a diagnostic pulse of a predetermined force and the diagnostic pulse is communicated to the sensing probe 18. In this way the sensing probe 18 can transmit the diagnostic pulse into the tooth 22. The diagnostic unit 26 senses resistance to the diagnostic pulse when the sensing probe 18 transmits the diagnostic pulse into the tooth 22. Moreover, the diagnostic unit 26 analyzes the resistance to the diagnostic pulse for determining a magnitude of the resistance. The diagnostic unit 26 compares the magnitude of the resistance to a pre-determined diagnostic range of magnitude. In this way the diagnostic unit 26 can determine the health of the tooth 22 in a precise, non-subjective manner. The diagnostic unit 26 includes a display 27 for displaying numbers indicating the value of the magnitude of resistance to visually communicate the health of the tooth 22 to the dentist.

The diagnostic unit 26 comprises a housing 28 that has a front wall 30 and a perimeter wall 32. A control circuit 34 is integrated into the housing 28 and a conductor 36 is coupled between the perimeter wall 32 of the housing 28 and the second end 16 of the handle 12. The conductor 36 is in communication with the control circuit 34 and the conductor 36 is in communication with the sensing probe 18. Additionally, the conductor 36 is in communication with the activate button 24. The conductor 36 might include a force conducting section that is capable of transmitting the diagnostic pulse to the sensing probe 18. Additionally, the conductor 36 might include an electrically conductive section.

The diagnostic unit 26 includes a pulse generator 38 that is integrated into the housing 28. The pulse generator 38 is electrically coupled to the control circuit 34 and the pulse generator 38 is in communication with the conductor 36. The pulse generator 38 is turned on when the activate button 24 is depressed. Moreover, the pulse generator 38 generates the diagnostic pulse into the conductor 36 thereby facilitating the conductor 36 to communicate the diagnostic pulse to the sensing probe 18. The diagnostic pulse may have a force sufficient to cause a tooth 22 to displace slightly when the tooth 22 is not healthy but insufficient to displace a tooth 22 that is healthy. Additionally, the diagnostic pulse has a force that is sufficiently low enough to avoid damaging enamel and other structures of the tooth 22.

The diagnostic unit 26 includes a signal analyzer 40 that is integrated into the housing 28 and the signal analyzer 40 is electrically coupled to the control circuit 34. The signal analyzer 40 is in communication with the conductor 36 and the signal analyzer 40 analyzes the resistance to the diagnostic pulse for determining the magnitude of resistance. The signal analyzer 40 may be an electronic signal analyzer that is capable of determining the magnitude of resistance the tooth 22 poses to the diagnostic pulse.

The display 27 is coupled to the front wall 30 of the housing 28 such that the display 27 is visible to the dentist and the display 27 is electrically coupled to the control circuit 34. The display 27 displays indicia 44 comprising numbers 46 ranging between 1 and 10. Each of the numbers 46 corresponds to the predetermined diagnostic range of magnitude ranging between a minimum magnitude and a maximum magnitude. In this way the numbers 46 communicate the magnitude of resistance to the dentist to facilitate the dentist to diagnose the health of the tooth 22. The display 27 may be a liquid crystal display or other type of electronic display. Additionally, the diagnostic range of magnitude may be a range determined by dental health professionals.

The diagnostic unit 26 includes a data port 48 that is integrated into the perimeter wall 32 of the housing 28 such that the data port 48 can receive a data cable. The data port 48 is electrically coupled to the control circuit 34 thereby facilitating the data port 48 to download data to an external computer. The data port 48 may be a universal serial bus port or other type of data port. The diagnostic unit 26 includes a power button 50 that is movably integrated into the front wall 30 of the housing 28. The power button 50 is electrically coupled to the control circuit 34 and the power button 50 turns the control circuit 34 on and off. A power supply 52 is positioned inside the housing 28, the power supply 52 is electrically coupled to the control circuit 34 and the power supply 52 comprises at least one battery.

In use, the curved end 20 of the sensing probe 18 is positioned against a tooth 22 in the patient's mouth 23 at a desired location on the tooth 22. The activate button 24 is depressed and the pulse generator 38 generates the pulse into the sensing probe 18. Additionally, the signal analyzer 40 analyzes the resistance of the tooth 22 to the diagnostic pulse. The resistance is compared to the diagnostic range of magnitude and the display 27 displays the number 46, ranging between 1 and 10, that corresponds to the diagnostic range of magnitude. In this way the dentist can precisely diagnose the health of each tooth 22 in the patient's mouth 23.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A dental diagnostic assembly for measuring reacting of teeth to a measured force for determining the health of the teeth, said assembly comprising:
a handle being configured to be gripped by a dentist;
a sensing probe being coupled to said handle, said sensing probe having a curved end wherein said curved end is configured to be positioned against a tooth in a patient's mouth;
a diagnostic unit being in remote communication with said sensing probe, said diagnostic unit producing a diagnostic pulse of a predetermined force, said diagnostic pulse being communicated to said sensing probe wherein said sensing probe is configured to transmit the diagnostic pulse into the tooth, said diagnostic unit sensing resistance to said diagnostic pulse when said sensing probe transmits said diagnostic pulse into the tooth, said diagnostic unit analyzing said resistance to said diagnostic pulse for determining a magnitude of said resistance, said diagnostic unit comparing said magnitude of said resistance to a pre-determined diagnostic range of magnitude wherein said diagnostic unit is configured to determine the health of the tooth, said diagnostic unit including a display for displaying numbers indicating the value of said magnitude of resistance wherein said display is configured to visually communicate the health of the tooth to the dentist.

2. The assembly according to claim 1, wherein:
said handle has a first end, a second end and an outer surface extending between said first end and said second end, said handle being elongated between said first end and said second end;
said sensing probe extends away from said first end of said handle, said curved end being distally positioned with respect to said first end of said handle, said sensing probe being comprised of a rigid material thereby facilitating said sensing probe to transmit a force wherein said sensing probe is configured to transmit the force into the tooth when said sensing probe is positioned against the tooth; and
said assembly includes an activate button being movably integrated into said handle wherein said activate button is configured to be manipulated by a dentist.

3. The assembly according to claim 1, wherein said diagnostic unit comprises:
a housing having a front wall and a perimeter wall;
a control circuit being integrated into said housing;
a conductor being coupled between said perimeter wall of said housing and said second end of said handle, said conductor being in communication with said control circuit, said conductor being in communication with said sensing probe; and
a pulse generator being integrated into said housing, said pulse generator being electrically coupled to said control circuit, said pulse generator being in communication with said conductor, said pulse generator being turned on when said activate button is depressed, said pulse generator generating said diagnostic pulse into said conductor thereby facilitating said conductor to communicate said diagnostic pulse to said sensing probe.

4. The assembly according to claim 3, wherein:
said diagnostic unit includes a signal analyzer being integrated into said housing, said signal analyzer being electrically coupled to said control circuit, said signal analyzer being in communication with said conductor, said signal analyzer analyzing said resistance to said diagnostic pulse for determining said magnitude of resistance; and
said display is coupled to said front wall of said housing wherein said display is configured to be visible to the dentist, said display being electrically coupled to said control circuit, said display displaying indicia comprising numbers ranging between 1 and 10, each of said numbers corresponding to said predetermined diagnostic range of magnitude ranging between a minimum magnitude and a maximum magnitude wherein said indicia are configured to communicate said magnitude of resistance to the dentist to facilitate the dentist to diagnose the health of the tooth.

5. A dental diagnostic assembly for measuring reacting of teeth to a measured force for determining the health of the teeth, said assembly comprising:
a handle being configured to be gripped by a dentist, said handle having a first end, a second end and an outer surface extending between said first end and said second end, said handle being elongated between said first end and said second end;
a sensing probe being coupled to said handle, said sensing probe having a curved end wherein said curved end is configured to be positioned against a tooth in a patient's mouth, said sensing probe extending away from said first end of said handle, said curved end being distally positioned with respect to said first end of said handle, said sensing probe being comprised of a rigid material thereby facilitating said sensing probe to transmit a force wherein said sensing probe is configured to transmit the force into the tooth when said sensing probe is positioned against the tooth;
an activate button being movably integrated into said handle wherein said activate button is configured to be manipulated by a dentist;
a diagnostic unit being in remote communication with said sensing probe, said diagnostic unit producing a diagnostic pulse of a predetermined force, said diagnostic pulse being communicated to said sensing probe wherein said sensing probe is configured to transmit the diagnostic pulse into the tooth, said diagnostic unit sensing resistance to said diagnostic pulse when said sensing probe transmits said diagnostic pulse into the tooth, said diagnostic unit analyzing said resistance to said diagnostic pulse for determining a magnitude of said resistance, said diagnostic unit comparing said magnitude of said resistance to a pre-determined diagnostic range of magnitude wherein said diagnostic unit is configured to determine the health of the tooth, said diagnostic unit including a display for displaying numbers indicating the value of said magnitude of resistance wherein said display is configured to visually communicate the health of the tooth to the dentist, said diagnostic unit comprising:
a housing having a front wall and a perimeter wall;
a control circuit being integrated into said housing;
a conductor being coupled between said perimeter wall of said housing and said second end of said handle, said conductor being in communication with said control circuit, said conductor being in communication with said sensing probe, said conductor being in communication with said activate button;
a pulse generator being integrated into said housing, said pulse generator being electrically coupled to said control circuit, said pulse generator being in communication with said conductor, said pulse generator being turned on when said activate button is depressed, said pulse generator generating said diagnostic pulse into said conductor thereby facilitating said conductor to communicate said diagnostic pulse to said sensing probe;
a signal analyzer being integrated into said housing, said signal analyzer being electrically coupled to said control circuit, said signal analyzer being in communication with said conductor, said signal analyzer analyzing said resistance to said diagnostic pulse for determining said magnitude of resistance;
said display being coupled to said front wall of said housing wherein said display is configured to be visible to the dentist, said display being electrically coupled to said control circuit, said display displaying indicia comprising numbers ranging between 1 and 10, each of said numbers corresponding to said predetermined diagnostic range of magnitude ranging between a minimum magnitude and a maximum magnitude wherein said indicia are configured to communicate said magnitude of resistance to the dentist to facilitate the dentist to diagnose the health of the tooth;
a data port being integrated into said perimeter wall of said housing wherein said data port is configured to receive a data cable, said data port being electrically coupled to said control circuit thereby facilitating said data port to download data to an external computer;
a power button being movably integrated into said front wall of said housing, said power button being electrically coupled to said control circuit, said power button turning said control circuit on and off; and
a power supply being positioned inside said housing, said power supply being electrically coupled to said control circuit, said power supply comprising at least one battery.
